(19) United States
(12) Patent Application Publication
(10) Pub. No.: US 2021/0106300 A1
(43) Pub. Date: Apr. 15, 2021

(54) MULTI-PARAMETRIC WEIGHTED MULTI-BED PET ACQUISITION AND RECONSTRUCTION

(71) Applicant: Siemens Medical Solutions USA, Inc., Malvern, PA (US)

(72) Inventors: Girish Bal, Knoxville, TN (US); Vladimir Y. Panin, Knoxville, TN (US); Frank Kehren, Knoxville, TN (US); Bernard Bendriem, Knoxville, TN (US)

(21) Appl. No.: 16/598,393

(22) Filed: Oct. 10, 2019

(51) Int. Cl. A61B 6/00; G06T 11/00; A61B 6/04; A61B 6/03

(52) U.S. Cl. CPC A61B 6/5241; G06T 11/005; A61B 6/0407; G06T 2210/41; A61B 6/037; A61B 6/5247; A61B 6/032; A61B 6/5282

(57) ABSTRACT

A method for performing a multi-bed scan includes receiving scanner-specific information including scanner sensitivity and receiving patient-specific information including attenuation. An attenuation-weighted sensitivity profile is calculated based on the scanner sensitivity and the attenuation. Individual bed scan times for each bed in a multi-bed study is calculated based on the attenuation-weighted sensitivity profile and the multi-bed scan is performed using the calculated individual bed scan times.

### TECHNICAL FIELD

This application relates generally to multi-bed imaging and, more particularly, to scanning protocols for multi-bed nuclear imaging.

### BACKGROUND

Certain nuclear imaging, such as positron emission tomography (PET) imaging, has a limited field of view (FOV) and cannot capture whole body images. In order to perform whole body imaging, multiple PET images are captured at multiple positions with respect to a patient (e.g., beds). Current multi-bed scanning systems use a simple ramp or cosine weighting function to stitch multi-bed images. A half-bed overlap can be assumed between beds to allow for stitching and alignment of multi-bed scans. In current systems, a scan having a lower signal-to-noise ratio can overwhelm data from a scan having a larger signal-to-noise ratio in an overlap region.

For scanners having a long axial field of view (FOV), the sensitivity of each bed in a multi-bed scan will be increased, due to the increased FOV. In addition, the overlap for each bed can be increased for long axial FOV scanners. Increased bed overlap can result in increased influence due to lower signal-to-noise ratio scans, attenuation, or sensitivity changes.

### SUMMARY

In some embodiments, a computer-implemented method is disclosed. The computer-implemented method includes the steps of receiving scanner-specific information including scanner sensitivity, receiving patient-specific information including attenuation, calculating an attenuation-weighted sensitivity profile based on the scanner sensitivity and the attenuation, calculating individual bed scan time for each bed in a multi-bed study based on the attenuation-weighted sensitivity profile, and performing the multi-bed scan using the calculated individual bed scan times.

In some embodiments, a computer-implemented method is disclosed. The computer-implemented method includes the steps of receiving a nuclear image data set obtained by a first image modality, calculating a sensitivity (S), attenuation (A), and scan time (T) weighting factors, and generating a multi-bed reconstruction of the nuclear image data set using a combination of the sensitivity (S), attenuation (A), and scan time (T) weighting factors (referred to as “SAT weighting factors” in combination).

In some embodiments, a system is disclosed. The system includes a first imaging modality and a computer. The computer is configured to receive scanner-specific information including scanner sensitivity, receive patient-specific information including attenuation, calculate an attenuation-weighted sensitivity profile based on the scanner sensitivity and the attenuation, calculate individual bed scan time for each bed in a multi-bed study based on the attenuation-weighted sensitivity profile, and perform the multi-bed scan using the calculated individual bed scan times.

### BRIEF DESCRIPTION OF THE DRAWINGS

The features and advantages of the present invention will be more fully disclosed in, or rendered obvious by the following detailed description of the preferred embodiments, which are to be considered together with the accompanying drawings wherein like numbers refer to like parts and further wherein:

FIG. 1 illustrates a nuclear imaging system, in accordance with some embodiments.

FIG. 2 illustrates a block diagram of a computer system, in accordance with some embodiments.

FIG. 3 is a graph illustrating a number of voxels per slice in a multi-bed CT scan having a 60 cm FOV, in accordance with some embodiments.

FIG. 4 is a full-body image generated from the multi-bed CT scan illustrated in FIG. 3, in accordance with some embodiments.

FIG. 5 illustrates reconstructions for each independent bed used in the three-bed reconstruction of FIG. 4, in accordance with some embodiments.

FIG. 6 is a flowchart illustrating a method of optimizing a scan protocol for a multi-bed study, in accordance with some embodiments.

FIG. 7 includes a plurality of graphs illustrating a total sensitivity per slice for three-bed scans conducted using different scan times for each bed, in accordance with some embodiments.

FIG. 8 includes a plurality of graphs illustrating the mean attenuation weighted sensitivity for each three-bed scan represented in FIG. 7, in accordance with some embodiments.

FIG. 9 illustrates a method of generating a stitched reconstructed image using attenuation-weighted sensitivity, in accordance with some embodiments.

FIG. 10A illustrates a stitched reconstructed image generated using a ramp function.

FIG. 10B illustrates a stitched reconstructed image generated using attenuation-weighted sensitivity weighting factors, in accordance with some embodiments.

FIG. 11A illustrates a clinical image generated using a ramp function.

FIG. 11B illustrates a clinical stitched reconstructed image generated using sensitivity, attenuation, and scan time (SAT) weighting factors, in accordance with some embodiments.

### DETAILED DESCRIPTION

The description of the preferred embodiments is intended to be read in connection with the accompanying drawings, which are to be considered part of the entire written description of this invention. The drawing figures are not necessarily to scale and certain features of the invention can be shown exaggerated in scale or in somewhat schematic form in the interest of clarity and conciseness. In this description, relative terms such as “horizontal,” “vertical,” “up,” “down,” “top,” “bottom,” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. In the claims, means-plus-function clauses, if used, are intended to cover structures described, suggested, or rendered obvious by the written description or drawings for performing the recited function, including not only structure equivalents but also equivalent structures.

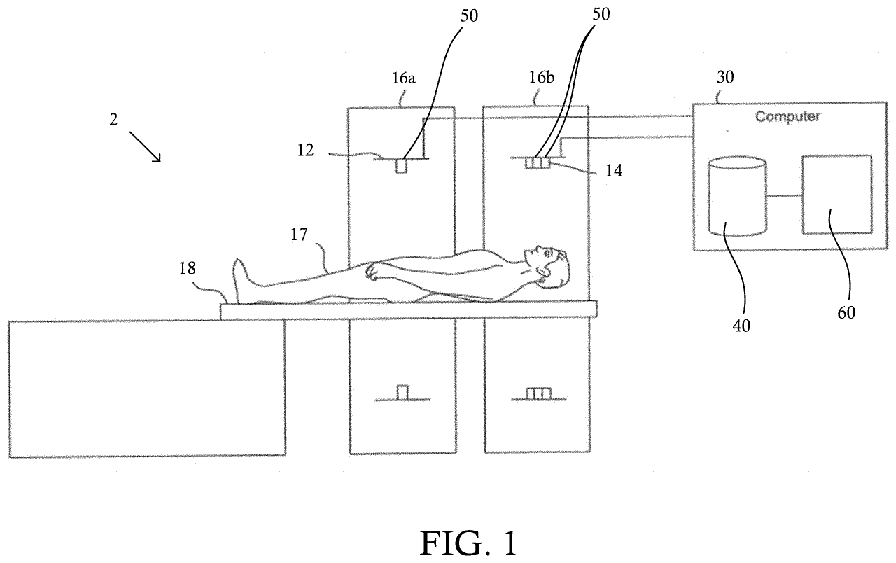

FIG. 1 illustrates one embodiment of a nuclear imaging system 2. The nuclear imaging system 2 includes a scanner for at least a first modality 12 provided in a first gantry 16a. The first modality 12 can include any suitable modality, such as, for example, a computed-tomography (CT) modality, a positron-emission tomography (PET) modality, a single-photon emission computerized tomography (SPECT) modality, etc. The first modality 12 can include a long axial FOV scanner or a short axial FOV scanner. A patient 17 lies on a movable patient bed 18 that is movable with respect to the first gantry 16a. In some embodiments, the nuclear imaging system 2 includes a scanner for a second modality 14 provided in a second gantry 16b. The second modality 14 can be any suitable imaging modality, such as, for example, a CT modality, a PET modality, a SPECT modality and/or any other suitable imaging modality. The second modality 14 can include a long axial FOV scanner or a short axial FOV scanner. Each of the first modality 12 and/or the second modality 14 can include one or more detectors 50 configured to detect an annihilation photon, gamma ray, and/or other nuclear imaging event. Although embodiments are discussed herein including a first modality 12 and/or a second modality 14, it will be appreciated that the disclosed systems and methods can be applied to any number of modalities either sequentially and/or independent of other modalities.

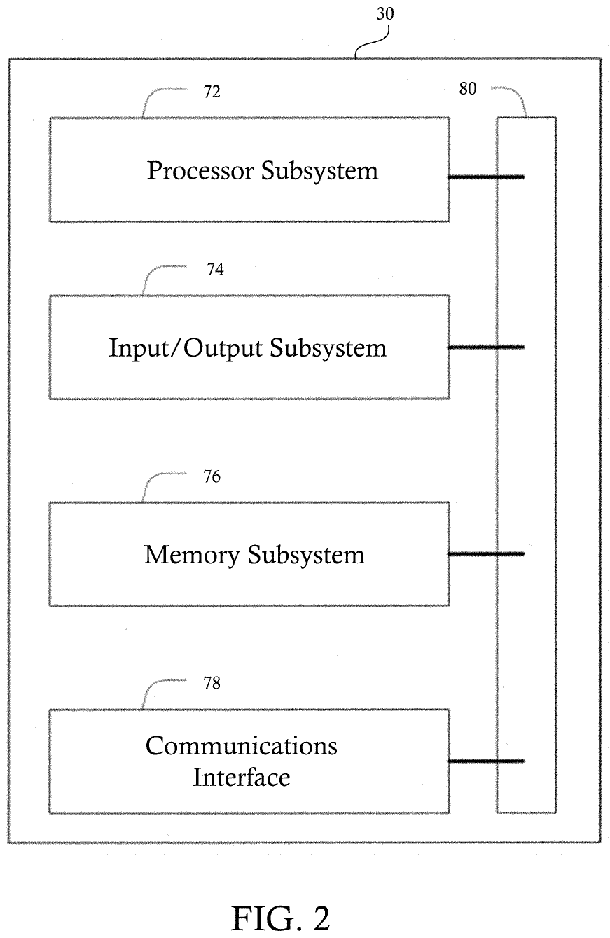

Scan data from the first modality 12, the second modality 14, and/or additional modalities is stored at one or more computer databases 40 and processed by one or more computer processors 60 of a computer system 30. The graphical depiction of computer system 30 in FIG. 1 is provided by way of illustration only, and computer system 30 can include one or more separate computing devices, for example, as described with respect to FIG. 2. The scan data can be provided by the first modality 12, the second modality 14, and/or can be provided as a separate data set, such as, for example, from a memory coupled to the computer system 30. The computer system 30 can include one or more processing electronics for processing a signal received from one of the plurality of detectors 50.

Certain scan protocols require scanning regions larger than the axial length (e.g., single bed position) of a modality 12, 14. Systems and methods of optimizing a scanning protocol using a plurality of parameters based on scanner and/or patient data are disclosed. For example, in some embodiments, scanner sensitivity, patient attenuation, number of counts per slice or region of interest, etc. can be used to optimize a scan protocol. In some embodiments, after scan data is acquired, it is necessary to reconstruct the data from multiple beds to generate a single reconstructed image. Systems and methods of reconstruction (i.e., stitching) configured to weight (or optimize) voxels with higher signal to noise ratio higher than voxels having lower signal to noise ratio during stitching. The disclosed systems and methods can be used for a single modality, e.g., modality 12, 14, and/or multiple modalities, for example, sequentially, independently, etc.

FIG. 2 illustrates a computer system 30 configured to implement one or more processes, in accordance with some embodiments. The system 30 is a representative device and can include a processor subsystem 72, an input/output subsystem 74, a memory subsystem 76, a communications interface 78, and a system bus 80. In some embodiments, one or more than one of the system 30 components can be combined or omitted such as, for example, not including an input/output subsystem 74. In some embodiments, the system 30 can comprise other components not shown in FIG. 2. For example, the system 30 can also include, for example, a power subsystem. In other embodiments, the system 30 can include several instances of a component shown in FIG. 2. For example, the system 30 can include multiple memory subsystems 76. For the sake of conciseness and clarity, and not limitation, one of each component is shown in FIG. 2.

The processor subsystem 72 can include any processing circuitry operative to control the operations and performance of the system 30. In various aspects, the processor subsystem 72 can be implemented as a general purpose processor, a chip multiprocessor (CMP), a dedicated processor, an embedded processor, a digital signal processor (DSP), a network processor, an input/output (I/O) processor, a media access control (MAC) processor, a radio baseband processor, a co-processor, a microprocessor such as a complex instruction set computer (CISC) microprocessor, a reduced instruction set computing (RISC) microprocessor, and/or a very long instruction word (VLIW) microprocessor, or other processing device. The processor subsystem 72 also can be implemented by a controller, a microcontroller, an application specific integrated circuit (ASIC), a field programmable gate array (FPGA), a programmable logic device (PLD), and so forth.

In various aspects, the processor subsystem 72 can be arranged to run an operating system (OS) and various applications. Examples of an OS comprise, for example, operating systems generally known under the trade name of Apple OS, Microsoft Windows OS, Android OS, Linux OS, and any other proprietary or open source OS. Examples of applications comprise, for example, network applications, local applications, data input/output applications, user interaction applications, etc.

In some embodiments, the system 30 can include a system bus 80 that couples various system components including the processing subsystem 72, the input/output subsystem 74, and the memory subsystem 76. The system bus 80 can be any of several types of bus structure(s) including a memory bus or memory controller, a peripheral bus or external bus, and/or a local bus using any variety of available bus architectures including, but not limited to, 9-bit bus, Industrial Standard Architecture (ISA), Micro-Channel Architecture (MSA), Extended ISA (EISA), Intelligent Drive Electronics (IDE), VESA Local Bus (VLB), Peripheral Component Interconnect Card International Association Bus (PCMCIA), Small Computers Interface (SCSI) or other proprietary bus, or any custom bus suitable for computing device applications.

In some embodiments, the input/output subsystem 74 can include any suitable mechanism or component to enable a user to provide input to system 30 and the system 30 to provide output to the user. For example, the input/output subsystem 74 can include any suitable input mechanism, including but not limited to, a button, keypad, keyboard, click wheel, touch screen, motion sensor, microphone, camera, etc.

In some embodiments, the input/output subsystem 74 can include a visual peripheral output device for providing a display visible to the user. For example, the visual peripheral output device can include a screen such as, for example, a Liquid Crystal Display (LCD) screen. As another example, the visual peripheral output device can include a movable display or projecting system for providing a display of content on a surface remote from the system 30. In some embodiments, the visual peripheral output device can include a coder/decoder, also known as Codecs, to convert digital media data into analog signals. For example, the visual peripheral output device can include video Codecs, audio Codecs, or any other suitable type of Codec.

The visual peripheral output device can include display drivers, circuitry for driving display drivers, or both. The visual peripheral output device can be operative to display content under the direction of the processor subsystem 72. For example, the visual peripheral output device can be able to play media playback information, application screens for application implemented on the system 30, information regarding ongoing communications operations, information regarding incoming communications requests, or device operation screens, to name only a few.

In some embodiments, the communications interface 78 can include any suitable hardware, software, or combination of hardware and software that is capable of coupling the system 30 to one or more networks and/or additional devices. The communications interface 78 can be arranged to operate with any suitable technique for controlling information signals using a desired set of communications protocols, services or operating procedures. The communications interface 78 can include the appropriate physical connectors to connect with a corresponding communications medium, whether wired or wireless.

Vehicles of communication comprise a network. In various aspects, the network can include local area networks (LAN) as well as wide area networks (WAN) including without limitation Internet, wired channels, wireless channels, communication devices including telephones, computers, wire, radio, optical or other electromagnetic channels, and combinations thereof, including other devices and/or components capable of/associated with communicating data. For example, the communication environments comprise in-body communications, various devices, and various modes of communications such as wireless communications, wired communications, and combinations of the same.

Wireless communication modes comprise any mode of communication between points (e.g., nodes) that utilize, at least in part, wireless technology including various protocols and combinations of protocols associated with wireless transmission, data, and devices. The points comprise, for example, wireless devices such as wireless headsets, audio and multimedia devices and equipment, such as audio players and multimedia players, telephones, including mobile telephones and cordless telephones, and computers and computer-related devices and components, such as printers, network-connected machinery, and/or any other suitable device or third-party device.

Wired communication modes comprise any mode of communication between points that utilize wired technology including various protocols and combinations of protocols associated with wired transmission, data, and devices. The points comprise, for example, devices such as audio and multimedia devices and equipment, such as audio players and multimedia players, telephones, including mobile telephones and cordless telephones, and computers and computer-related devices and components, such as printers, network-connected machinery, and/or any other suitable device or third-party device. In various implementations, the wired communication modules can communicate in accordance with a number of wired protocols. Examples of wired protocols can include Universal Serial Bus (USB) communication, RS-232, RS-422, RS-423, RS-485 serial protocols, FireWire, Ethernet, Fibre Channel, MIDI, ATA, Serial ATA, PCI Express, T-1 (and variants), Industry Standard Architecture (ISA) parallel communication, Small Computer System Interface (SCSI) communication, or Peripheral Component Interconnect (PCI) communication, to name only a few examples.

Accordingly, in various aspects, the communications interface 78 can include one or more interfaces such as, for example, a wireless communications interface, a wired communications interface, a network interface, a transmit interface, a receive interface, a media interface, a system interface, a component interface, a switching interface, a chip interface, a controller, and so forth. When implemented by a wireless device or within wireless system, for example, the communications interface 78 can include a wireless interface comprising one or more antennas, transmitters, receivers, transceivers, amplifiers, filters, control logic, and so forth.

In various aspects, the communications interface 78 can provide data communications functionality in accordance with a number of protocols. Examples of protocols can include various wireless local area network (WLAN) protocols, including the Institute of Electrical and Electronics Engineers (IEEE) 802.xx series of protocols, such as IEEE 802.11a/b/g/n/ac, IEEE 802.16, IEEE 802.20, and so forth. Other examples of wireless protocols can include various wireless wide area network (WWAN) protocols, such as GSM cellular radiotelephone system protocols with GPRS, CDMA cellular radiotelephone communication systems with 1×RTT, EDGE systems, EV-DO systems, EV-DV systems, HSDPA systems, and so forth. Further examples of wireless protocols can include wireless personal area network (PAN) protocols, such as an Infrared protocol, a protocol from the Bluetooth Special Interest Group (SIG) series of protocols (e.g., Bluetooth Specification versions 5.0, 6, 7, legacy Bluetooth protocols, etc.) as well as one or more Bluetooth Profiles, and so forth. Yet another example of wireless protocols can include near-field communication techniques and protocols, such as electro-magnetic induction (EMI) techniques. An example of EMI techniques can include passive or active radio-frequency identification (RFID) protocols and devices. Other suitable protocols can include Ultra Wide Band (UWB), Digital Office (DO), Digital Home, Trusted Platform Module (TPM), ZigBee, and so forth.

In some embodiments, at least one non-transitory computer-readable storage medium is provided having computer-executable instructions embodied thereon, wherein, when executed by at least one processor, the computer-executable instructions cause the at least one processor to perform embodiments of the methods described herein. This computer-readable storage medium can be embodied in memory subsystem 76.

In some embodiments, the memory subsystem 76 can include any machine-readable or computer-readable media capable of storing data, including both volatile/non-volatile memory and removable/non-removable memory. The memory subsystem 8 can include at least one non-volatile memory unit. The non-volatile memory unit is capable of storing one or more software programs. The software programs can contain, for example, applications, user data, device data, and/or configuration data, or combinations therefore, to name only a few. The software programs can contain instructions executable by the various components of the system 30.

In various aspects, the memory subsystem 76 can include any machine-readable or computer-readable media capable of storing data, including both volatile/non-volatile memory and removable/non-removable memory. For example, memory can include read-only memory (ROM), random-access memory (RAM), dynamic RAM (DRAM), Double-Data-Rate DRAM (DDR-RAM), synchronous DRAM (SDRAM), static RAM (SRAM), programmable ROM (PROM), erasable programmable ROM (EPROM), electrically erasable programmable ROM (EEPROM), flash memory (e.g., NOR or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, disk memory (e.g., floppy disk, hard drive, optical disk, magnetic disk), or card (e.g., magnetic card, optical card), or any other type of media suitable for storing information.

In one embodiment, the memory subsystem 66 can contain an instruction set, in the form of a file for executing various methods, such as methods including A/B testing and cache optimization, as described herein. The instruction set can be stored in any acceptable form of machine readable instructions, including source code or various appropriate programming languages. Some examples of programming languages that can be used to store the instruction set comprise, but are not limited to: Java, C, C++, C #, Python, Objective-C, Visual Basic, or .NET programming In some embodiments a compiler or interpreter is comprised to convert the instruction set into machine executable code for execution by the processing subsystem 72.

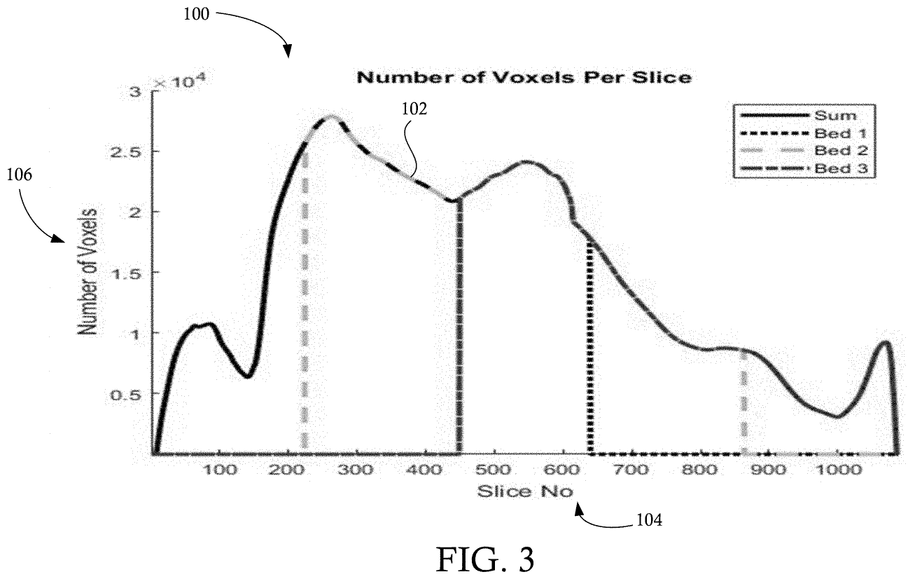

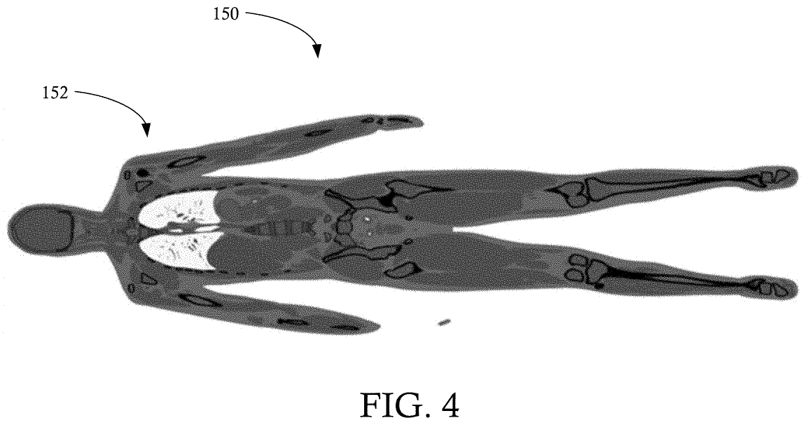

FIG. 3 is a graph 100 illustrating a number of voxels per slice 102 in a multi-bed CT scan having a 60 cm FOV, in accordance with some embodiments. The chart 100 includes an X-axis 104 showing a slice number for a multi-bed scan and a Y-axis 106 showing the number of voxels. The term voxel, as used herein, refers to a value in three-dimensional space, e.g., a position in a three-dimensional image, such as a reconstruction generated from nuclear imaging data. The term slice, as used herein, refers to a portion of the body that is imaged. As illustrated in FIG. 3, a multi-bed scan can include over 1000 slices for a full-body reconstruction. The number of voxels per slice indicates the number of three-dimensional values (e.g., three-dimensional pixels) within a predetermined portion of a reconstruction. FIG. 4 is a full-body image 150 generated from the multi-bed CT scan represented by the graph 100 in FIG. 3, in accordance with some embodiments.

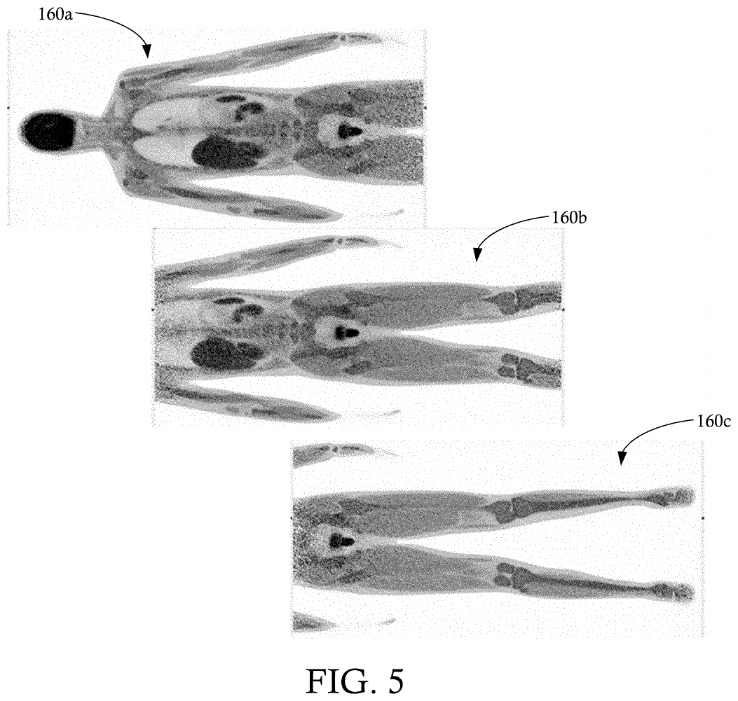

As shown in FIG. 3, the number of voxels per slice 102 increases as the scanner moves from the head of a patient 152 to the chest and decreases over the remainder of the patient 152, i.e., the concentration of the CT scan was around the chest and abdomen of the patient 152. FIG. 3 illustrates a three-bed scan with a first bed extending from slice 0 to about slice 625, a second bed extending from slice 210 to about slice 835 and a third bed extending from about slice 415 to the last slice. The illustrated embodiment includes a voxel overlap region of about 415 slices for each bed. FIG. 5 illustrates reconstructions 160a-160c for each independent bed used in the three-bed reconstruction 150 of FIG. 4. The reconstructions 160a-160c are arranged to show the overlapping slices. As shown in FIG. 5, the overlap between each subsequent bed reconstruction 160a-160c is greater than the half-bed overlap used in current systems. In contrast to current systems, the reconstructions 160a-160c are arranged have a majority/minority overlap, such as, for example, 60-80% overlap, 65-75% overlap, 25-35% overlap, and/or any other suitable overlap.

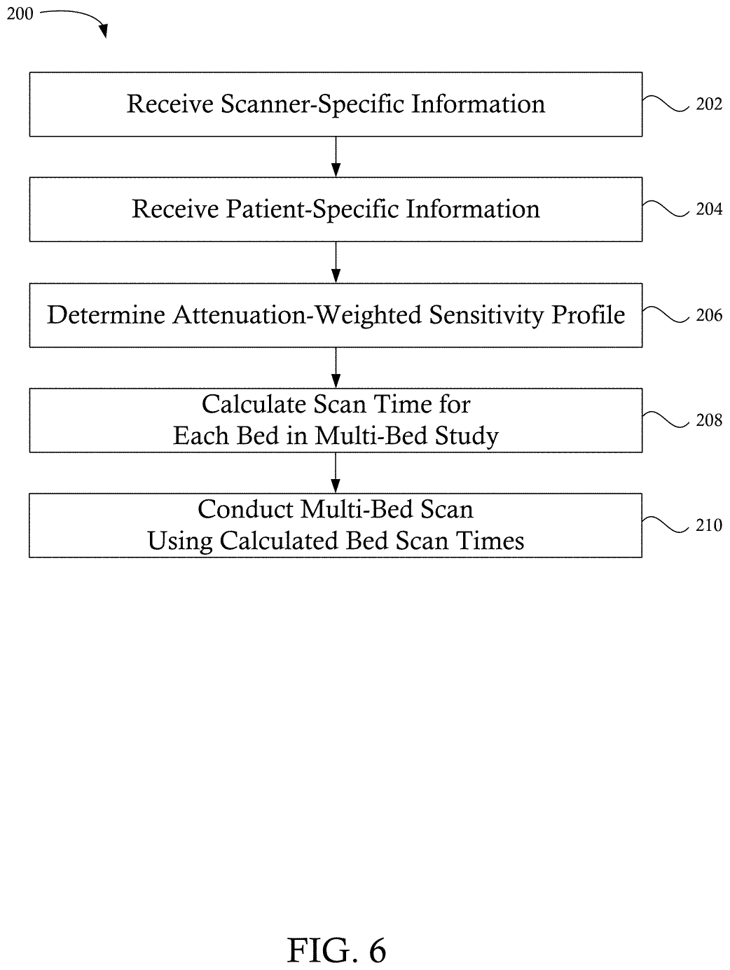

In some embodiments, sensitivity of a scanner and/or an attenuation profile can be used to determine an optimal scan time per bed (i.e., a scan protocol) in a multi-bed study. FIG. 6 is a flowchart 200 illustrating a method of optimizing a scan protocol for one of a multi-bed study or a continuous bed motion (CBM) study, in accordance with some embodiments. At step 202, scanner-specific information is received. The scanner-specific information can be received by any suitable system, such as, for example, the computer 30. The scanner-specific information can be received from an external source, such as, for example, a second computer/memory system external to the computer 30, received from an internal source such as an internal memory module, detachable memory module, etc., and/or received using any other suitable data transfer mechanism. The scanner-specific information includes sensitivity information for the specific scanner to be used in a multi-bed study. The scanner-specific information can include additional information, such as, for example, FOV length, modality type, and/or any other suitable scanner information.

At step 204, patient-specific information is received. The patient-specific information can be received from any suitable source, such as, for example, an internal memory source, an external memory source, and/or any other suitable source using any suitable data transfer mechanism. The patient-specific information includes a patient-specific attenuation profile indicating an attenuation rate for the patient. The patient-specific attenuation profile can be based on prior and/or concurrent scan data (e.g., calculated from one or more prior and/or concurrent nuclear scans, such as a CT scan, MRI Scan, etc.) and/or calculated from other patient-specific parameters, such as height, weight, age, and/or any other suitable patient-specific parameters. For example, in some embodiments, a CT scan is conducted prior to and/or concurrently with a PET scan. An attenuation map can be generated from the CT scan and forward projected into a sinogram space to generate an attenuation map to be used in a reconstruction with respect to the PET scan.

At step 206, an attenuation-weighted sensitivity profile is determined based on the scanner-specific sensitivity information and the patient-specific attenuation profile. The attenuation-weighted sensitivity profile is scanner-specific and patient-specific. The attenuation-weighted sensitivity profile can be generated by back-projecting the sensitivity and attenuation coefficient factors, using artificial intelligence approaches, and/or using any other suitable process. As used herein, the term attenuation-weighted sensitivity profile refers to any profile calculated using one or more a scanner sensitivity, patient attenuation, total measured counts, slice by slice measured counts, true, prompts, scatter, random, model-derived parameters and/or other suitable parameters.

At step 208, a bed scan time for each bed in a multi-bed scan and/or each chunk in a continuous bed motion scan is calculated based on the attenuation-weighted sensitivity profile. As used herein, the term bed scan time is used to refer individual bed scan times in multi-bed scan and scan chunk times in a continuous bed motion scan. The scan time for each bed can be configured to provide a predetermined mean attenuation weighted sensitivity for the multi-bed scan. For example, in some embodiments, the scan time of each bed is configured to provide a uniform mean attenuation weighted sensitivity for the entire multi-bed scan. The uniform mean attenuation weighted sensitivity provides a uniform scan across the length of the patient. As another example, in some embodiments, the predetermined mean attenuation weighted sensitivity includes a higher sensitivity at a predetermined portion of a patient, such as the chest, abdomen, pelvis, etc. The scan time of each bed can independent and/or can be related to the scan time of one or more additional beds in a multi-bed study.

In some embodiments, the adjusted scan time for each bed is calculated using one or more additional parameters, such as, for example, total measured counts, slice by slice measured counts, true, prompts, scatter, random, and/or other suitable parameters. The parameters can be identified using one or more artificial-intelligence based systems, such as, for example, one or more trained models. In some embodiments, the use of sensitivity, attenuation, and measured counts (e.g., counts per slice) are configured to generate adjusted scan times for each bed that maximize the signal-to-noise ratio (SNR) for each bed to provide a predetermined noise profile, such as a uniform noise profile for the entire scan, a user defined region specific SNR, and/or other suitable predetermined noise profile.

As used herein, the term true refers to a detected event or line of effect having a specifically identified location within a space defined by an imaging modality. As used herein, the term prompts refers to events that have a statistical probability of being identified as a specific event, such as, for example, a true, scatter, or random. As used herein, the term scatter refers to a detected event or line of effect that has changed from its origin due to an interaction with, for example, one or more anatomical structures. As used herein, the term random refers to a detected event originating outside of a detection space defined by an imaging modality, such as the first imaging modality 12. As used herein, the term model-derived parameters is used to refer parameters derived from and/or identified through the use of one or more trained artificial intelligence models.

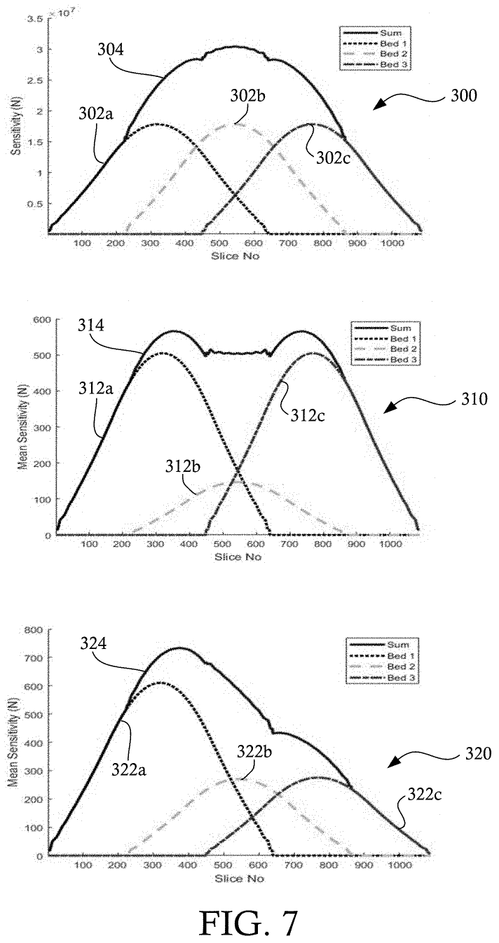

FIG. 7 includes a plurality of graphs 300, 310, 320 each illustrating a mean sensitivity per slice for a three-bed scan conducted using different scan times for each bed, in accordance with some embodiments. The graph 300 corresponds to a three-bed scan having a fixed, equal time period for each bed (referred to herein as a same time per bed scan), the graph 310 corresponds to a three-bed scan having scan times configured to provide a uniform sensitivity profile for the entire multi-bed scan (referred to herein as a uniform sensitivity scan), and the graph 320 corresponds to a three bed scan having scan times configured to provide a uniform attenuation-weighted sensitivity profile for the entire scan (referred to herein as a uniform NA scan). For example, in the illustrated embodiment, each of the multi-bed scans has a total scan time of 10 minute. For the same time per bed scan, each bed has a scan time of about 3.33 minutes. For the uniform sensitivity scan, the first and third bed each have a scan time of about 4.365 minutes and the second bed has a scan time of about 1.27 minutes. For the uniform NA scan, the first bed has a scan time of about 5.2783 minutes, the second bed has a scan time of about 2.3387 minutes, and the third bed has a scan time of about 2.383 minutes. It will be appreciated that the scan times provided herein are provided only as examples and that the individual scan times will vary based on scanner sensitivity profiles, patient attenuation profiles, total scan times, modality, and/or other parameters, and it will be appreciated that any suitable time period and/or total time period can be used for any of the discussed scans.

With respect to the same time per bed scan 300, the sensitivity profile 302a-302c for each bed includes a curve that starts at zero prior to the bed scan time, increases to a maximum sensitivity at the center of the bed/bed scan time, and declines to zero at the end of the bed scan time. The maximum sensitivity for each sensitivity profile 302a-302c is the same. A full-scan sensitivity profile 304 for the entire same time per bed scan 300 is equal to the first bed sensitivity profile 302a from time t₀ to time (t₁) at which the second bed overlaps the first bed. The full-scan sensitivity profile 304 increases to a peak at time t₂ at which all of the beds overlap and decreases from the peak to zero, with the rate of decrease increasing at a time t₃, at which the second third bed does not overlap with the second bed.

With respect to the uniform sensitivity scan 310, the sensitivity profile 312a-312c for each bed indicates a higher sensitivity for the first bed 312a and the third bed 312c. The sensitivity of the second bed 312b is reduced (due to the lower scan time). The full-scan sensitivity profile 314 for the uniform sensitivity scan 310 follows the sensitivity profile 312a of the first bed from time t₀ to a time t₁ at which the second bed overlaps the first. The full-scan sensitivity profile 314 is generally flat from time t₁ to time t₂ (with peaks occurring at the overlap between beds 1 and 2 and beds 2 and 3). The full-scan sensitivity profile is equal to the third bed sensitivity profile 312c from time t₂ to time t₃.

With respect to the uniform NA scan 320, the sensitivity profile 322a-322c for each bed is variable based on the scan time for each bed, with the sensitivity profile 322a of the first bed having the highest peak (corresponding to a longer scan time) and the sensitivity profiles 322b, 322c of the second and third beds being relatively equal (corresponding to relatively equal scan times). The full-scan sensitivity profile 324 follows the sensitivity profile 322a of the first bed from time t₀ to time t₁ at which the second bed overlaps the first bed. The full-scan sensitivity profile 324 increases to a peak at time t₂ and decreases from the peak at a relatively steady rate from time t₂ to time t₃.

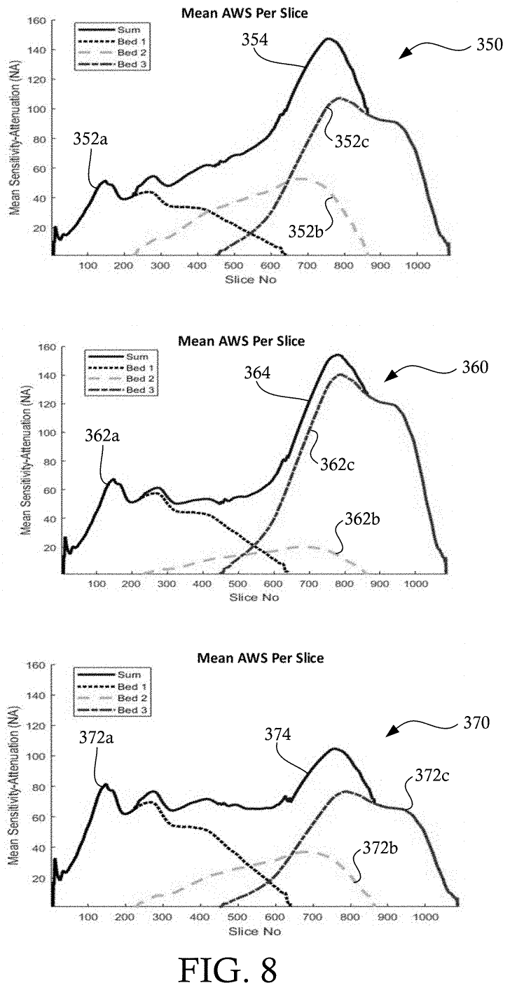

Although the same time per bed scan 300 and the uniform sensitivity scan 310 each show a relatively high sensitivity for slices corresponding to the chest and abdomen area, the mean attenuation-weighted sensitivity for each of the same time per bed scan 300 and the uniform sensitivity scan 310 is relatively low due to the effects of attenuation in these areas. FIG. 8 includes graphs 350, 360, 370 illustrating the mean attenuation weighted sensitivity for each three-bed scan represented in FIG. 7, in accordance with some embodiments. The graph 350 corresponds to the same time per bed scan, the graph 360 corresponds to the uniform sensitivity scan, and the graph 370 corresponds to the uniform NA scan.

In each graph 350-370, the mean attenuation-weighted sensitivity for each bed is determined by dividing an attenuation-weighted sensitivity for each bed by the number of voxels per slice 102 (as illustrated in FIG. 3). A mean attenuation weighted sensitivity for the full scan is calculated by summing the individual mean attenuation weighted sensitivities for each bed.

Although the attenuation weighted sensitivity 302a-302c for each bed in the same time per bed scan 300 (see FIG. 7) looks similar, the mean attenuation weighted sensitivity 352a, 352b for the first bed and the second bed in the same time per bed scan 350 is lower than the mean attenuation weighted sensitivity 352c for the third bed. As a result, the total mean attenuation weighted sensitivity 354 for the same time per bed scan 350 covering the upper and pelvic regions of the body (e.g., from about slice 0 to about slice 600) is very low as compared to the mean attenuation weighted sensitivity 354 for the lower region of the body (e.g., from about slice 600 to about slice 1100).

Similarly, although the sensitivity profile 314 of the uniform sensitivity scan 310 is substantially uniform (see FIG. 7), the mean attenuation-weighted sensitivity 362a, 362b for the first bed and the second bed in uniform sensitivity scan 360 is substantially less than the mean attenuation-weighted sensitivity 362c for the third bed. As a result, the total mean attenuation-weighted sensitivity 364 for the uniform sensitivity scan 360 is lower in the upper and pelvic regions of the body as compared to the lower region of the body.

In contrast to the same time per bed scan 350 and the uniform sensitivity scan 360, the uniform NA scan 370 includes a total mean attenuation-weighted sensitivity 374 that is generally uniform for the upper, pelvic, and lower regions of the body. The mean attenuation-weighted sensitivity 372a-372c for each bed is varied to provide the generally uniform total mean attenuation-weighted sensitivity 374.

With reference again to FIG. 6, at step 210, the scan time for each bed is provided to a scanner, such as the scanner 2 illustrated in FIG. 1, and a multi bed scan is performed using the calculated scan times. The multi-bed scan can include a single scanning modality 12 and/or multiple scanning modalities 12, 14. Scan times configured to provide a uniform NA scan similarly provide a more uniform noise profile for reconstruction of images from the scan data. In some embodiments, a uniform NA scan reduces the time spent scanning low attenuation regions of a patient and increases the time spent scanning high attenuation regions of the patient.

As one example, a patient can be selected for a three-bed multi-bed cardiac study. The patient can be overweight or obese and therefore have a large amount of attenuation in the chest. The attenuation-weighted sensitivity profile will show a lower attenuation-weighted sensitivity in the chest area. The scan time for the first bed and/or the second bed (e.g., the beds overlapping the chest area) is increased and the scan time of the third bed (e.g., the bed not overlapping the chest area) reduced to increase the scan time in the high attenuation regions of the patient, increasing the attenuation-weighted sensitivity of those areas. The scan time for each bed can be related to the scan time for one or more other beds, the total scan time, and/or can be independently calculated.

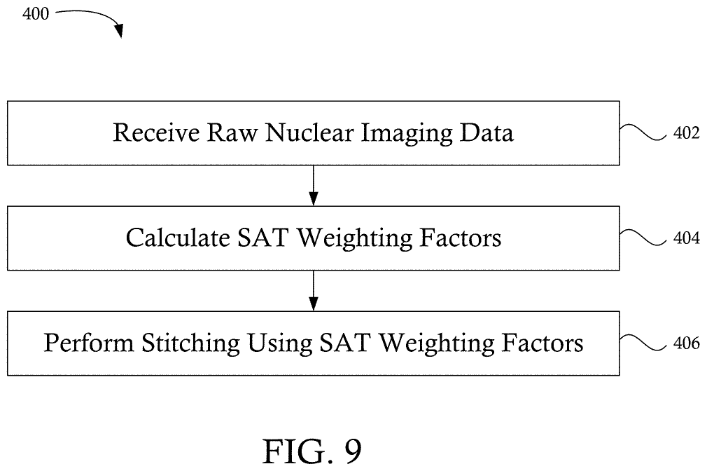

FIG. 9 illustrates a method 400 of generating a stitched reconstructed image using attenuation-weighted sensitivity, in accordance with some embodiments. At step 402, scan data is received. The scan data can be received from any suitable system, such as, for example, a scanning modality 12, 14, a memory module coupled to and/or in signal communication with a system, such as computer system 30, and/or any other suitable source. The scan data can be obtained from any suitable imaging modality, such as, for example, a PET image modality.

At step 404, sensitivity, attenuation, and scan time (SAT) weighting factors are calculated and, at step 406, stitching is performed using the SAT weighting factors. In some embodiments, the SAT weighting factors are calculated simultaneous with the stitching operation. For example, in some embodiments, stitching is performed according to the equation:

$${I}_{j , s} ( x ) = \frac{{\sum }_{b = 1}^{B} ( {I}_{j , b} ( x ) \cdot {S}_{j , b} ( x ) \cdot {A}_{j , b} ( x ) \cdot {T}_{j , b} ( x ) )}{{\sum }_{b = 1}^{B} ( {S}_{j , b} ( x ) \cdot {A}_{j , b} ( x ) \cdot {T}_{j , b} ( x ) )}$$

where I_{j,s}(x) is the j^{th} voxel in the final stitched image, I_{j,b}(x) is reconstructed image for bed ‘b’, S_{j,b}(x) is sensitivity of the scanner/scan, A_{j,b}(x) is attenuation and T_{j,b}(x) is scan time for that bed/frame/gate at location j. The terms for sensitivity and attenuation can be in the image space or in a projection domain. In various embodiments, one or more additional parameters, such as, for example, total measured counts, slice by slice measured counts, true, prompts, scatter, random, and/or other suitable parameters can be used independently and/or in conjunction with one or more of a scanner sensitivity, patient attenuation, and/or scan time to generate the SAT weighting factors.

Similarly, in some embodiments, stitching can be performed according to the equation:

$${I}_{j , s} ( x ) = \frac{{\sum }_{b = 1}^{B} {\sum }_{g = 1}^{G} ( {I}_{j , b , g} ( x ) \cdot {S}_{j , b , g} ( x ) \cdot {A}_{j , b , g} ( x ) \cdot {T}_{j , b , g} ( x ) )}{{\sum }_{b = 1}^{B} {\sum }_{g = 1}^{G} ( {S}_{j , b , g} ( x ) \cdot {A}_{j , b , g} ( x ) \cdot {T}_{j , b , g} ( x ) )}$$

where I_{j,s}(x) is a j^{th} voxel in the reconstruction, I_{j,b,g}(x) is a reconstructed image for a bed ‘b’ and a gate (or frame) ‘g’, S_{j,b,g}(x) is sensitivity of the first image modality, A_{j,b,g}(x) is attenuation and T_{j,b},g(x) is scan time for the bed ‘b’ and gate ‘g’ at location j. The term gate is used herein to refer to one or more time slots in a respiratory, cardiac, and/or dynamic tracer uptake phases, random motion by the patient, and/or other movement of the patient. Gates can be repetitive, random and/or have a time based decay factor. One or more parameters, such as, for example, trues, randoms, scatter, prompts, model-derived parameters, etc. can vary during one or more gates and are incorporated into the above equation.

In some embodiments, the stitching equation discussed above gives a greater weight to voxels with higher SNR during stitching. A uniform mean sensitivity is generated and provides a more uniform noise profile. In contrast, current stitching processes use 100% of bed 1 information at the midpoint of bed 1, even if an overlapping bed (e.g., bed 2) has a higher SNR for the same location. The disclosed method and equation can be used for step-and-shoot image reconstruction and/or continuous bed motion reconstruction.

In some embodiments, if multiple frames or gates are present in the scan data, e.g., in the case of continuous motion or dynamic varying counts, the SAT weighting factors of each bed, frame, and/or gate can be determined. A motion vector can be added to a predetermined combination of the SAT weighting factors for each frame. The motion vector can be further used in the stitching step to facilitate stitching of different beds, frames, and/or gates. In some embodiments, such as embodiments including dynamic studies, the sensitivity (S) term can be affected by dead time. A combination of SAT weighting factors can be applied during stitching to incorporate dead time information during the reconstruction and/or multi-bed stitching.

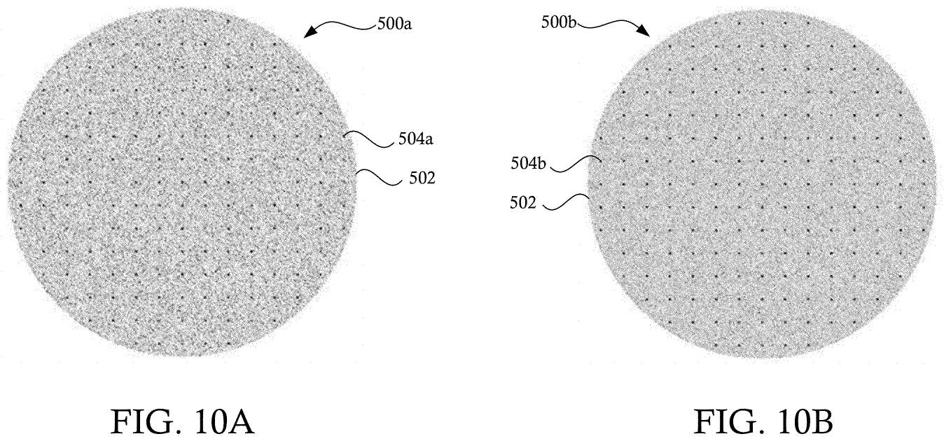

FIG. 10A illustrates a reconstruction image 500a of a test rod 502 generated using a traditional reconstruction method. FIG. 10B illustrates a reconstruction image 500b of the test rod 502 using the method 400 of generating a stitched reconstructed image using attenuation-weighted sensitivity, in accordance with some embodiments. As shown in FIGS. 10A and 10B, the method 100 provides improved lesion detectability over the traditional method. The reconstruction 500b generated using the SAT weighting factors has a high SNR and includes a clearer image with well-defined lesion points 504b. In contrast, the traditional reconstruction 500a includes a low SNR, resulting in a blurry image with stretched, concealed, and/or otherwise unclear lesion points 504a. Although embodiments are discussed herein including the use of sensitivity and attenuation for the stitching algorithm, it will be appreciated that a multi-parametric stitching process can be based on measured and/or determined parameters using an artificial intelligence based method.

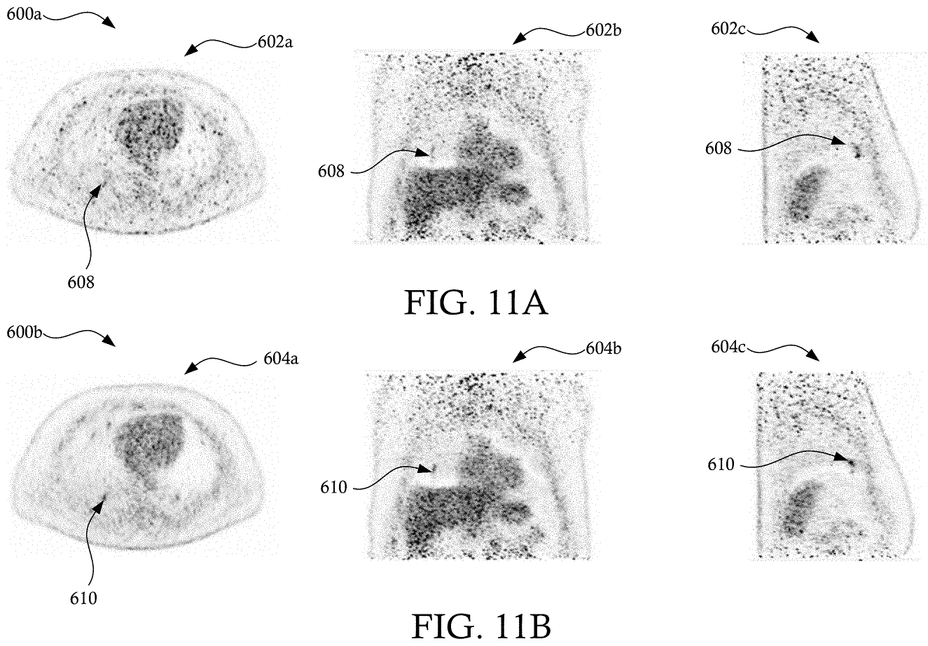

FIG. 11A illustrates a set of clinical reconstruction images 600a of nuclear imaging data generated using a traditional reconstruction method. FIG. 11B illustrates a set of clinical reconstruction images 600b of the nuclear imaging data generated using the method 400 of generating a stitched reconstructed image using attenuation-weighted sensitivity, in accordance with some embodiments. As shown in FIG. 11B, the nuclear imaging data includes a lesion 610 that is visible in each of the three different reconstruction images 604a-604c. The high SNR provided by the method 400 allows the lesion 610 to be easily identified. In contrast, as shown in FIG. 11A, the traditional reconstruction method produces images having a lower SNR such that the lesion 608 is barely visible in the first and second reconstruction images 602a, 602b and is misplaced in the third reconstruction image 602c.

As illustrated in FIGS. 10A-11B, the SNR of a reconstructed image is better when using an SAT-based weighting function, e.g., method 400, as compared to a traditional ramping and/or cosine based weighting. In the case of a mismatch between bed data, the bed having the higher SNR is given a greater weight, resulting in higher standard uptake value (SUV) peaks, improved lesion detectability, and less noise in the overlap regions. The proposed method 400 is capable of adjusting certain voxels in the image space that receive contributions from multiple beds and/or for varying scan times to favor the scan data (e.g., bed) having a higher SNR. The proposed method 400 can be used for generating reconstructions from step-and-scan and/or continuous-bed motion scans.

In a first embodiment, a computer-implemented method includes the steps of receiving scanner-specific information including scanner sensitivity, receiving patient-specific information including attenuation, calculating an attenuation-weighted sensitivity profile based on the scanner sensitivity and the attenuation, calculating bed scan time for each bed in a multi-bed study based on the attenuation-weighted sensitivity profile, and performing the multi-bed scan using the calculated bed scan times.

The bed scan time for each bed can be calculated using at least one total measured counts or slice by slice measured counts. The attenuation can include an attenuation map generated from a computed tomography scan. The individual bed scan time for each bed can be configured to provide a uniform total mean attenuation-weighted sensitivity for the multi-bed scan. The individual bed scan time for each bed can be configured to maximize a signal-to-noise ratio (SNR) for each bed and provide a substantially uniform noise profile for the multi-bed scan. The individual bed scan time can be configured to provide a greater mean attenuation-weighted sensitivity in a first region corresponding to a first portion of a patient as compared to a mean attenuation-weighted sensitivity in a second region.

In a second embodiment, a computer implemented method includes the steps of receiving a nuclear image data set obtained by a first image modality, calculating a sensitivity (S), attenuation (A), and scan time (T) weighting factors, and generating a multi-bed reconstruction of the nuclear image data set using a combination of the SAT weighting factors.

The reconstruction can be generated according to the equation:

$${I}_{j , s} ( x ) = \frac{{\sum }_{b = 1}^{B} ( {I}_{j , b} ( x ) \cdot {S}_{j , b} ( x ) \cdot {A}_{j , b} ( x ) \cdot {T}_{j , b} ( x ) )}{{\sum }_{b = 1}^{B} ( {S}_{j , b} ( x ) \cdot {A}_{j , b} ( x ) \cdot {T}_{j , b} ( x ) )}$$

where I_{j,s}(x) is a j^{th} voxel in the reconstruction, I_{j,b}(x) is a reconstructed image for a bed ‘b’, S_{j,b}(x) is sensitivity of the first image modality, A_{j,b}(x) is attenuation and T_{j,b}(x) is scan time for the bed ‘b’ at location j. The combination of the SAT weighting factors can be configured to provide a greater weight to voxels with a higher signal-to-noise ratio (SNR) during reconstruction. The SAT weighting factors can be configured to provide a uniform mean sensitivity and a uniform noise profile for the reconstruction.

In some embodiments, computer-implemented method of the first embodiment can be used to calculate a scan time prior to performing a scan. One or more of the calculated scan times can be adjusted, such as, for example, by a user. A scan can be performed using the adjusted scan times and a reconstruction generated using the computer-implemented method of the second embodiment.

In a third embodiment, a non-transitory computer-readable medium includes instructions which, when executed by a processor, cause the processor to carry out one or more of the computer implemented methods disclosed herein, such as the computer-implemented method of the first embodiment, the computer implemented method of the second embodiment, or any variation thereof

In a fourth embodiment, a system is disclosed. The system includes a first imaging modality and a computer. The computer is configured to receive scanner-specific information including scanner sensitivity, receive patient-specific information including attenuation, calculate an attenuation-weighted sensitivity profile based on the scanner sensitivity and the attenuation, calculate individual bed scan time for each bed in a multi-bed study based on the attenuation-weighted sensitivity profile, and perform the multi-bed scan using the calculated individual bed scan times.

The individual bed scan time for each bed can be calculated using at least one of total measured counts or slice by slice measured counts. The attenuation can include an attenuation map generated from a computed tomography scan. The individual bed scan time for each bed can be configured to provide a uniform total mean attenuation-weighted sensitivity for the multi-bed scan. The individual bed scan time for each bed can be configured to maximize a signal-to-noise ratio (SNR) for each bed and provide a uniform noise profile for the multi-bed scan. The individual bed scan time can be configured to provide a greater mean attenuation-weighted sensitivity in a first region corresponding to a first portion of a patient as compared to a mean attenuation-weighted sensitivity in a second region.

The computer can be further configured to receive an adjustment of at least one bed scan time calculated for the multi-bed study prior to performing the multi-bed study, receive a nuclear image data set from the first imaging modality corresponding to the multi-bed study, calculate SAT weighting factors, and generate a reconstruction of the nuclear image data set using the SAT weighting factors.

The reconstruction can be generated according to the equation:

$${I}_{j , s} ( x ) = \frac{{\sum }_{b = 1}^{B} {\sum }_{g = 1}^{G} ( {I}_{j , b , g} ( x ) \cdot {S}_{j , b , g} ( x ) \cdot {A}_{j , b , g} ( x ) \cdot {T}_{j , b , g} ( x ) )}{{\sum }_{b = 1}^{B} {\sum }_{g = 1}^{G} ( {S}_{j , b , g} ( x ) \cdot {A}_{j , b , g} ( x ) \cdot {T}_{j , b , g} ( x ) )}$$

where I_{j,s}(x) is a j^{th} voxel in the reconstruction, I_{j,b}(x) is a reconstructed image for a bed ‘b’, S_{j,b,g}(x) is sensitivity of the first image modality, A_{j,b,g}(x) is attenuation and T_{j,b},g(x) is scan time for the bed ‘b’ and gate ‘g’ at location j. The SAT weighting factors can be configured to provide a greater weight to voxels with a higher signal-to-noise ratio (SNR) during reconstruction. The SAT weighting factors can be configured to provide optimized signal to noise ratio in the reconstruction.

Although the subject matter has been described in terms of exemplary embodiments, it is not limited thereto. Rather, the appended claims should be construed broadly, to include other variants and embodiments, which can be made by those skilled in the art.

What is claimed is:

1. A computer-implemented method, comprising:
receiving scanner-specific information including scanner sensitivity;
receiving patient-specific information including attenuation;
calculating an attenuation-weighted sensitivity profile based on the scanner sensitivity and the attenuation;
calculating a bed scan time for each bed in a multi-bed study based on the attenuation-weighted sensitivity profile; and
performing the multi-bed scan using the calculated bed scan times.

2. The computer-implemented method of claim 1, wherein the bed scan time for each bed is calculated using at least one additional parameter selected from the group consisting of: total measured counts, slice by slice measured counts, true, prompts, scatter, randoms, and model-derived parameters.

3. The computer-implemented method of claim 1, wherein the bed scan time for each bed is configured to optimize measured counts for at least one predetermined region.

4. The computer-implemented method of claim 1, wherein the individual bed scan time for each bed is configured to provide a uniform total mean attenuation-weighted sensitivity for the multi-bed scan.

5. The computer-implemented method of claim 1, wherein the individual bed scan time for each bed is configured to maximize a signal-to-noise ratio (SNR) for each bed and provide a substantially uniform noise profile for the multi-bed scan.

6. The computer-implemented method of claim 1, wherein the individual bed scan time is configured to provide a greater mean attenuation-weighted sensitivity in a first region corresponding to a first portion of a patient as compared to a mean attenuation-weighted sensitivity in a second region.

7. A computer-implemented method, comprising
receiving a nuclear image data set obtained by a first image modality;
calculating sensitivity (S), attenuation (A), and scan time (T) weighting factors; and
generating a multi-bed reconstruction of the nuclear image data set using a combination of the sensitivity (S), attenuation (A), and scan time (T) (SAT) weighting factors.

8. The computer-implemented method of claim 7, wherein the reconstruction is generated according to the equation:
$${I}_{j , s} ( x ) = \frac{{\sum }_{b = 1}^{B} ( {I}_{j , b} ( x ) \cdot {S}_{j , b} ( x ) \cdot {A}_{j , b} ( x ) \cdot {T}_{j , b} ( x ) )}{{\sum }_{b = 1}^{B} ( {S}_{j , b} ( x ) \cdot {A}_{j , b} ( x ) \cdot {T}_{j , b} ( x ) )}$$
where I_{j,s}(x) is a j^{th} voxel in the reconstruction, I_{j,b}(x) is a reconstructed image for a bed ‘b’, S_{j,b}(x) is sensitivity of the first image modality, A_{j,b}(x) is attenuation and T_{j,b}(x) is scan time for the bed ‘b’ at location j.

9. The computer-implemented method of claim 7, comprising performing multi-bed stitching to generate a stitched image using a combination of the SAT weighting factors configured to provide a greater weight to voxels with a higher signal-to-noise ratio (SNR) during multi-bed stitching.

10. The computer-implemented method of claim 7, wherein the SAT weighting factors are configured to provide a uniform mean sensitivity and a uniform noise profile for the reconstruction.

11. A system, comprising:
a first imaging modality; and
a computer configured to:
receive scanner-specific information including scanner sensitivity;
receive patient-specific information including attenuation;
calculate an attenuation-weighted sensitivity profile based on the scanner sensitivity and the attenuation;
calculate individual bed scan time for each bed in a multi-bed study based on the attenuation-weighted sensitivity profile; and
perform the multi-bed scan using the calculated individual bed scan times.

12. The system of claim 11, wherein the bed scan time for each bed is calculated using at least one additional parameter selected from the group consisting of: total measured counts, slice by slice measured counts, true, prompts, scatter, randoms, and model-derived parameters.

13. The system of claim 11, wherein the attenuation comprises an attenuation map generated from a computed tomography scan.

14. The system of claim 11, wherein the individual bed scan time for each bed is configured to provide a uniform total mean attenuation-weighted sensitivity for the multi-bed scan.

15. The system of claim 11, wherein the individual bed scan time for each bed is configured to maximize a signal-to-noise ratio (SNR) for each bed and provide a uniform noise profile for the multi-bed scan.

16. The system of claim 11, wherein the individual bed scan time for each bed comprises one or more frames, wherein each frame comprises one or more gates, and wherein each of the one or more frames and the one or more gates includes a predetermined attenuation-weighted sensitivity profile.

17. The system of claim 16, wherein the computer is configured to:
receive an adjustment of at least one bed scan time calculated for the multi-bed study prior to performing the multi-bed study;
receive a nuclear image data set from the first imaging modality corresponding to the multi-bed study for each of the one or more frames or the one or more gates;
calculate first sensitivity (S), attenuation (A), and scan time (T) (SAT) weighting factors; and
generate a reconstruction of the nuclear image data set using the SAT weighting factors for each of the one or more frames or the one or more gates.

18. The system of claim 17, wherein the reconstruction is generated according to the equation:
$${I}_{j , s} ( x ) = \frac{{\sum }_{b = 1}^{B} {\sum }_{g = 1}^{G} ( {I}_{j , b , g} ( x ) \cdot {S}_{j , b , g} ( x ) \cdot {A}_{j , b , g} ( x ) \cdot {T}_{j , b , g} ( x ) )}{{\sum }_{b = 1}^{B} {\sum }_{g = 1}^{G} ( {S}_{j , b , g} ( x ) \cdot {A}_{j , b , g} ( x ) \cdot {T}_{j , b , g} ( x ) )}$$
where I_{j,s}(x) is a j^{th} voxel in the reconstruction, I_{j,b}(x) is a reconstructed image for a bed ‘b’, S_{j,b,g}(x) is sensitivity of the first image modality, A_{j,b,g}(x) is attenuation and T_{j,b},g(x) is scan time for the bed ‘b’ and gate ‘g’ at location j.

19. The system of claim 17, wherein the computer is configured to perform a multi-bed reconstruction based on second SAT weighting factors.

20. The system of claim 17, wherein the first SAT weighting factors are configured to provide optimized signal to noise ratio in the reconstruction.
